(19) United States
(12) Patent Application Publication
(10) Pub. No.: US 2023/0390493 A1
(43) Pub. Date: Dec. 7, 2023

(54) Medicament Delivery Device

(71) Applicant: Shaily (UK) Ltd, Croydon Surrey (GB)

(72) Inventor: Anders Holmqvist, Saltsjo-Boo (SE)

(73) Assignee: Shaily (UK) Ltd, Croydon Surrey (GB)

(21) Appl. No.: 18/022,893

(22) PCT Filed: Nov. 11, 2021
(86) PCT No.: PCT/GB2021/052912

§ 371 Date: Feb. 23, 2023

(30) Foreign Application Priority Data
Nov. 13, 2020 (SE) 2051331-3

(51) Int. Cl. A61M 5/20

(52) U.S. Cl. CPC A61M 5/2033; A61M 5/20; A61M 2005/2013; A61M 2005/2073

(57) ABSTRACT

A medicament delivery device is provided that has a body, a medicament container provided with a medicament delivery member arranged in the body, and a drive unit having a plunger rod and a drive spring. The drive unit is operatively connected to the medicament container. An activator is operatively connected to the drive unit. A safety mechanism is operatively connected to the plunger rod. In an initial state of the device, the plunger rod is held with the drive spring in a tensioned state and a safety mechanism is in contact with, and prevents release of, the plunger rod. When the device is pressed against an injection site, the activator acts on the safety mechanism for moving the safety mechanism out of contact with the plunger rod. The activator releases the plunger rod, causing the plunger rod to act on the medicament container for delivery of medicament.

### TECHNICAL FIELD

The present invention relates to a medicament delivery device and in particular an auto-injector.

### BACKGROUND OF INVENTION

Medicament delivery devices have been on the market for many years for self-administration of drugs. Many of these are designed as auto-injectors with the aim that the end-user should be required to perform as few steps as possible in order to initiate an injection.

U.S. Pat. No. 3,797,489 discloses an auto-injector with a pre-tensioned drive spring operably connected to a plunger rod. In order to activate the device a safety cap with a safety pin is removed and the device is pressed against an injection site. A user then presses a button on the rear end of the device, which causes a syringe with a needle to move forward and perform a penetration. The release of the plunger rod will then cause an injection. A retraction spring will be tensioned, such that when the device is removed after injection, the syringe and the needle are moved back into the injector.

U.S. Pat. No. 5,681,291 discloses an auto-injector having a needle cover movable inside a housing of the device. When the injector is pressed against an injection site, the needle cover is moved rearwards, causing a manual penetration. The movement of the needle cover will further cause an activation of a drive spring acting on a plunger rod for performing an injection. When the device is removed from the injection site, a spring pushes the needle cover into a position covering the needle, in which position it is locked.

Even though many of these devices on the market have adequate functionality and robustness, there is always room for improvements.

### BRIEF SUMMARY OF INVENTION

The aim of the present invention is to provide a medicament delivery device with a high degree of robustness and reliable functionality and safety features.

This aim is obtained with a medicament delivery device according to the independent patent claim. Preferable embodiments of the invention form the subject of the dependent patent claims.

According to a main aspect, a medicament delivery device is provided comprising a housing, a medicament container provided with a medicament delivery member arranged in the housing, a drive unit comprising a plunger rod and a drive spring.

The drive unit may be operatively connected to the medicament container, and an activator operatively connected to the drive unit, as well as a safety mechanism operatively connected to the plunger rod.

In an initial state of the device, the plunger rod is held with the drive spring in a tensioned state and wherein the safety mechanism is in contact with and prevents release of the plunger rod, ensuring that the device cannot be unintentionally activated.

When the device is pressed against an injection site, the activator acts on the safety mechanism for moving the safety mechanism out of contact with the plunger rod, which activates the device.

Further, the activator acts on the plunger rod for releasing the plunger rod, causing the plunger rod to act on the medicament container for delivery of medicament at the injection site. Because the activator positively acts on the plunger rod (directly or through an intermediate element) to release the plunger rod, this ensures a distinct point of activation. The reliability of the release system may be improved because activation does not depend on components sliding under the force of the drive spring on surfaces that offer frictional resistance.

Thus, the activator, when the device is used for an injection, functions to both remove the safety mechanism and to initiate an injection.

According to another aspect, the activator may comprise a release sleeve provided with release members that are moved in contact with the plunger rod for releasing the plunger rod. A release sleeve is a robust and functional design when using release members.

The medicament delivery device may further comprise a needle cover designed to be moved into said body and moving release sleeve. Thus the needle cover and the release sleeve cooperate for the activation of the device.

Further, the plunger rod may comprise flexible arms, wherein the flexible arms may comprise protrusions that cooperate with ledges fixed in the device, and wherein the release members push on the protrusions for flexing the arms to release the plunger rod. In this regard, the safety mechanism may comprise a safety pin in contact with the flexible arms, preventing release of the plunger rod. The safety pin ensures that the device cannot be accidentally activated for instance during transport or when a user drops the device.

According to a further aspect, the safety pin may be comprised in the release sleeve, the safety pin being designed to move out of contact with the arms of the plunger rod when the release members are moved into contact with and push on the protrusions of the plunger rod. With this design, the release sleeve comprises several functions.

Further, the medicament delivery device may comprise an intermediary release member in contact with the protrusions of the plunger rod, wherein the release members of the release sleeve are pushing on the intermediary release member for releasing the plunger rod. The intermediary release member facilitates the design and function of the release mechanism.

According to a further aspect, the device may comprise a needle cover spring acting on the release sleeve, whereby the release sleeve and the needle cover are moved to a forward position with the needle cover covering the medicament delivery member after removal of the device from an injection site. Again the release sleeve and the needle cover act together for covering the needle after injection.

In order to ensure the safety against accidental needle sticks, the release sleeve may further comprise locking members designed to lock the release sleeve and the needle cover in the forward position. Again the release sleeve is designed with several functions. In this regard, the locking members may comprise openings, further comprising a spring housing attached to the body, the spring housing comprising flexible locking members arranged to flex into the openings.

According to a further aspect, the spring housing may be designed generally tubular and elongated, surrounding the drive spring for controlling buckling of the drive spring during injection, thus ensuring a reliable function of the device. The spring housing may comprise a central passage and wherein the ledges are provided adjacent the central passage. The spring housing may further comprise support members designed to contact and support a rear surface of the syringe. The spring housing may further comprise an audible/tactile member designed to be in contact with the drive spring and slide along the drive spring during injection, thereby creating audible/tactile information to a user. The spring housing is also designed with a number of functions.

According to a further aspect, the device may comprise a syringe holder, the syringe holder may comprise openings surrounded by support surfaces, that the body may be provided with openings, wherein the support surfaces are designed to snap into the openings of said body, thereby attaching the syringe holder to the body. The syringe holder may further comprise a seat designed for supporting a flange at a rear end of the syringe.

The medicament delivery device may further comprise a cap attachable to a front end of the device, the cap comprising flexible hooks engaging a needle shield covering the medicament delivery member, wherein removal of the cap removes the needle shield. In this regard, the cap may further comprise flexible hooks at a front area of the cap for holding the needle shield after removal.

These and other aspects of, and advantages with, the present invention will become apparent from the following detailed description of the invention and from the accompanying drawings.

### BRIEF DESCRIPTION OF DRAWINGS

In the following detailed description of the invention, reference will be made to the accompanying drawings, of which

FIGS. 1-2 show cross-sectional views of a first embodiment of a medicament delivery device according to the invention,

FIGS. 3-21 show different components and elements comprised in the device of FIGS. 1 and 2,

FIGS. 22-30 show different functional states of the device of FIGS. 1 and 2,

FIGS. 31-32 show cross-sectional views of a second embodiment of a medicament delivery device according to the invention, and

FIGS. 33-39 show different components and elements comprised in the device of FIGS. 31 and 32.

### DETAILED DESCRIPTION OF THE INVENTION

### Definitions

“Front” side of the device—Meaning the needle side of the device, where the needle comes out during injection.

“Rear” side—Opposite the side where the needle comes out during injection.

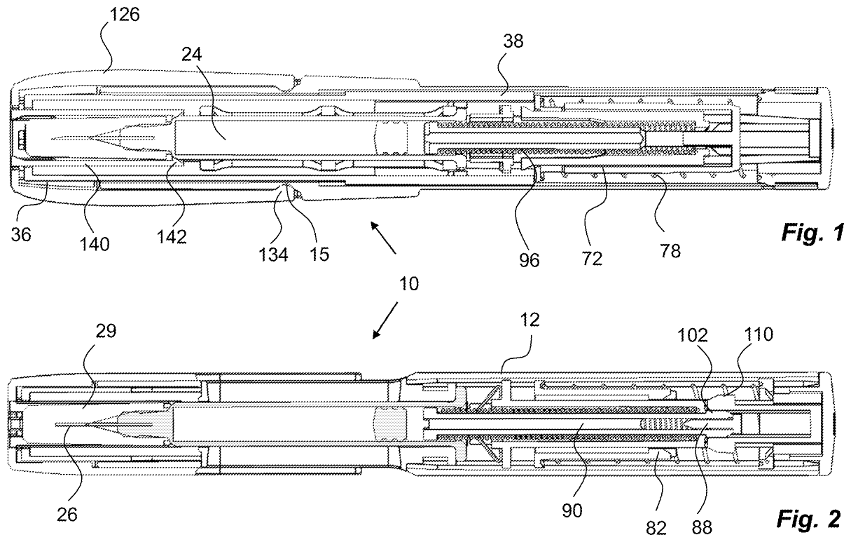

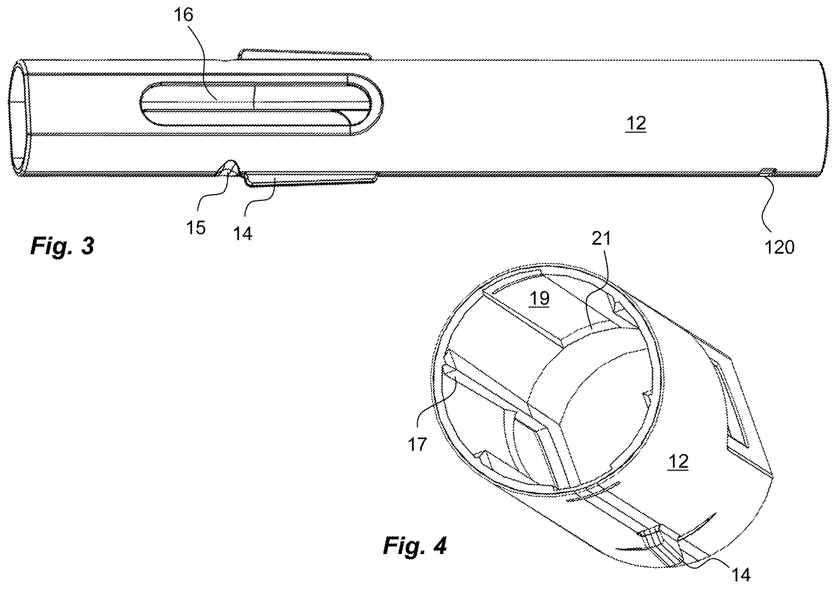

A first embodiment of a medicament delivery device 10 is shown in the FIGS. 1 to 30. The first embodiment is designed for a subcutaneous injection of a smaller volume of medicament, in the region of 1 ml. It comprises an elongated, generally tubular, body 12, FIGS. 3 and 4. The outer surface of the body 12 is provided with wings 14. The outer surface of the body 12 is also designed to hold a label with relevant information. The wings 14 on the outside of the body 12 act as both roll-stop and grip feature for a user. In front of the wings, recesses 15 are provided. The body 12 is further arranged with openings 16. The inner surface of the body 12 is further arranged with longitudinally extending guide ledges 17. Longitudinally extending guide grooves 19 are also arranged on the inner surface of the body 12, which guide grooves are provided with rearwardly facing stop ledges 21.

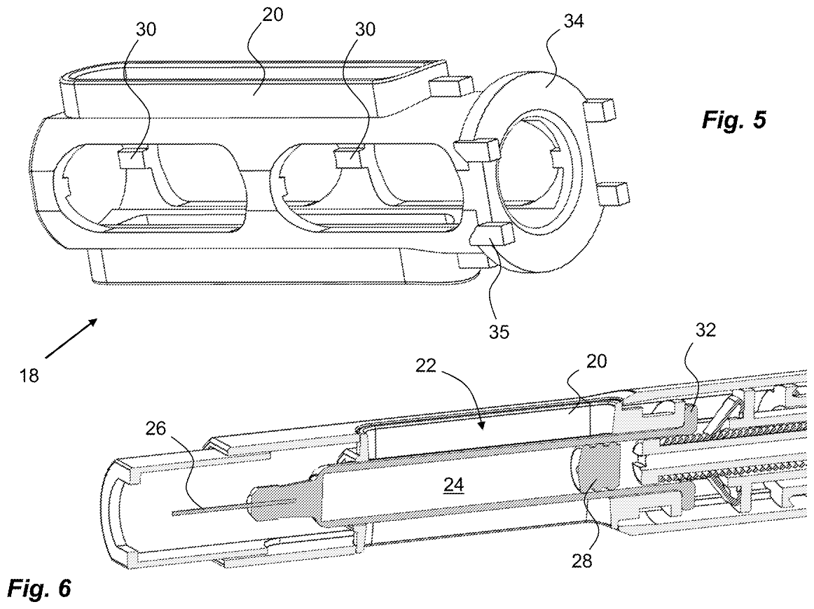

Inside the body 12 a syringe holder 18 is attached, FIGS. 5 and 6. The syringe holder 18 is arranged with outwardly extending walls 20 that form openings into the interior of the syringe holder 18, creating inspection windows 22 together with the opening 16 of the body 12. The walls 20 are designed to fit into the openings 16, wherein the syringe holder 18 is designed with flexible sections that will allow the walls to be compressed inwards during assembly and then snap into the openings 16, thereby locking the two components to each other as seen in FIG. 6.

Inside the syringe holder 18, a syringe 24, preferably of glass, with an injection needle 26 and a movable stopper 28 can be placed. Before use, the needle is preferably protected and kept sterile by a rigid needle shield, RNS, 29. The syringe with its content is visible through the inspection window openings 22. The syringe holder 18 is designed with guides 30 that support the syringe 24 radially. A flange 32 of the syringe 24 is supported by a seat 34 of the syringe holder 18 to prevent forward motion. The syringe holder 18 controls the position of the tip of the needle 26 by supporting the glass flange 32. This gives a defined length dimension with tolerances to consider. The surface of the seat 34 of the syringe holder 18 is designed to minimize glass breakage by being smooth and without single contact points. The seat 34 can be fitted with a rubber section, as a 2-K-mold or an O-ring sub-assembly, to smoothen the contact surface even more to avoid glass flange breakage. The seat 34 of the syringe holder 18 is further designed for a syringe “cut flange” which will hold the rotational orientation of that flange. However, a syringe with a “small round flange” can fit in the syringe holder as well. A small round flange has the advantage that it is stronger, and the risk of glass breakage is less. Further, the outer surface of the seat 34 is provided with planar surfaces and with protrusions 35 on each side for guide support (described below).

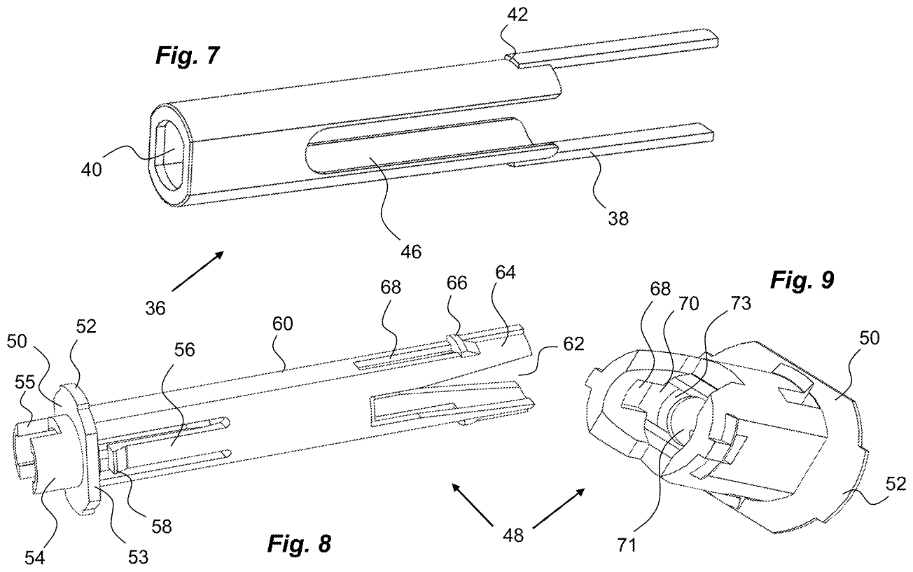

The device is further arranged with a needle cover 36, FIG. 7, which is a part of an activator of the device. The needle cover 36 is generally tubular and is arranged to extend a distance out of the front end of the body 12. The rear end of the needle cover 36 has two legs 38 extending in the rearward direction. An opening 40 in the front end of the needle cover 36 is small enough to prevent a sphere of diameter 12 mm to pass. This is a standard requirement to prevent accidental needle stick in fingers. The legs of the needle cover 36 are designed to fit into the guide grooves 19 of the body 12, wherein the stop ledges 21 interact with forwardly facing surfaces 42 of the legs 38, which will limit the movement of the needle cover 36 in the forward direction and prevent the needle cover 36 from falling out. Openings 46 are arranged in the sides of the needle cover 36 that allow movement of the needle cover around the inspection window walls 20 formed in the syringe holder 18. The inner surfaces of the legs 38 of the needle cover are planar and the width of the legs 38 is such that they are also guided by the protrusions 35 of the syringe holder 18. This also allows the legs 38 to be manufactured with a larger thickness, while the syringe holder 18 still has an edge to guide the rotation of a syringe flange, which is positive from a manufacturing point of view.

A generally tubular spring housing 48 is further disposed inside the body, FIGS. 8 and 9. At the front end, a guide plate 50 is disposed, provided with outwardly extending protrusions 52 that fit between the guide ledges 17 on the inner surface of the body 12, allowing axial movement of the spring housing 48 but rotational guide in relation to the body 12. The outer surface of the guide plate 50 is further designed with planar cut-outs 53 designed to guide the legs 38 of the needle cover 36. The guide plate 50 has forwardly extending features 54 that will contact the rear surface of the flange of the syringe 24 and keep it in its axial position, preventing it from moving more than a little in the rear direction. The features 54 could be a tubular body with cut-outs 55 as seen in FIG. 8. As an alternative, protruding ribs may be provided that extend forward and end a short distance before the flange of the syringe 24. The purpose of the feature 54 is to keep the syringe in its position if the syringe would have slipped backwards a distance from the syringe holder surface 34, so that it will not experience a high impact in the flange area depending on syringe acceleration and stop at activation.

The front area of the spring housing 48 is further provided with flexible arms 56 extending in the forward direction, where the free ends of the flexible arms 56 are provided with outwardly extending lock-out hooks 58, the function of which will be described below. The outer surface of the spring housing has longitudinally extending planar surfaces 60. The rear area of the spring housing is designed with two longitudinal slits 62, whereby two flexible arms 64 are created. At the rear ends of the arms 64, outwardly extending wedges 66 are arranged. In front of the wedges 66, elongated slits 68 are arranged. A transversal wall 70 is arranged inside the spring housing at the area where the slits 68 terminate in the forward direction. The transversal wall 70 is provided with a central passage 71 and ledges 73 are formed on the transversal wall 70 between the passage 71 and the slits 68, FIG. 9.

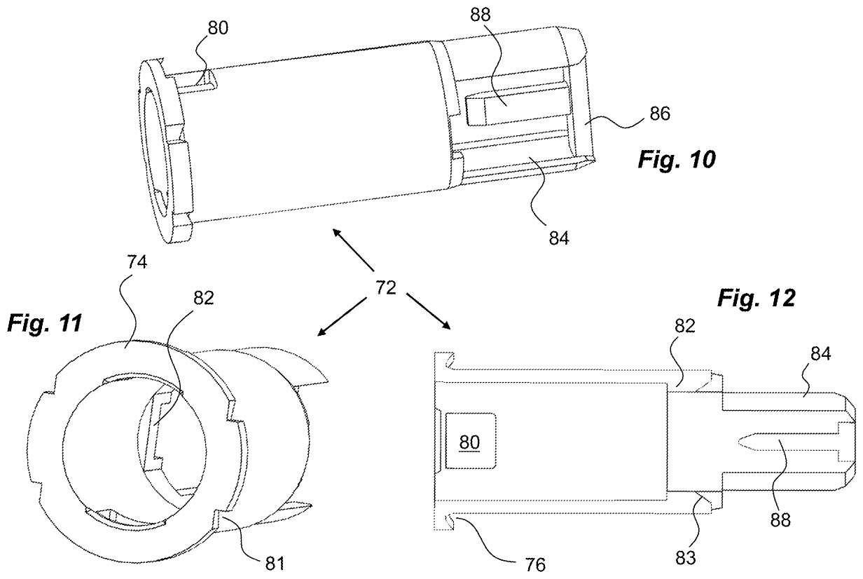

The device further comprises a release sleeve 72 that is a part of an activator as well as a safety mechanism of the device, FIGS. 10-12. The front section of the release sleeve 72 is generally tubular and has a front plate 74 that interacts with the end surfaces of the legs 38 of the needle cover 36 such that when the needle cover 36 is moved in the rear direction, so is the release sleeve 72. The release sleeve 72 has a spring seat 76 for a needle cover spring 78, FIG. 1. The needle cover spring 78 shall be stiff enough to act as a transportation safety mechanism, that together with the design of components of the device keeps the device safe and un-activated by vibrations and shocks that can occur during transportation. The seat 76 is deep enough to prevent the coil wire end from being pushed outside the spring diameter and jam or interfere with the body 12 during movement. The spring seat 76 has the release sleeve component's largest diameter, but is still small enough to avoid contact with the inside of the body 12.

The inside surface of the body 12 is part of guiding the needle cover spring 78 in a radial direction if/when the needle cover spring 78 is buckling away from the ideal axial position. The outer diameter of the front section of the release sleeve 72 acts as an inner guide for the needle cover spring 78 to prevent buckling. The front section is also arranged with openings 80 rear of the front plate 74. Cut-outs 81 are formed in the outer surface of the front plate, in which cut-outs 81 the guide ledges 17 of the body 12 fit, providing a rotational lock between the components but allowing axial movement of the release sleeve 72 in relation to the body 12. The inner surface of the release sleeve 72 is further provided with ledges 82 that are generally planar and are designed to slide against the planar surfaces 60 of the spring housing 48. The rear ends of the ledges 82 are provided with wedge-like surfaces 83. The rear end of the release sleeve 72 is designed with two elongated arms 84. Between the arms 84, a transversally extending beam 86 or bridge is provided. To the forwardly facing surface of the beam 86, an elongated safety pin 88 is provided, extending in the forward direction.

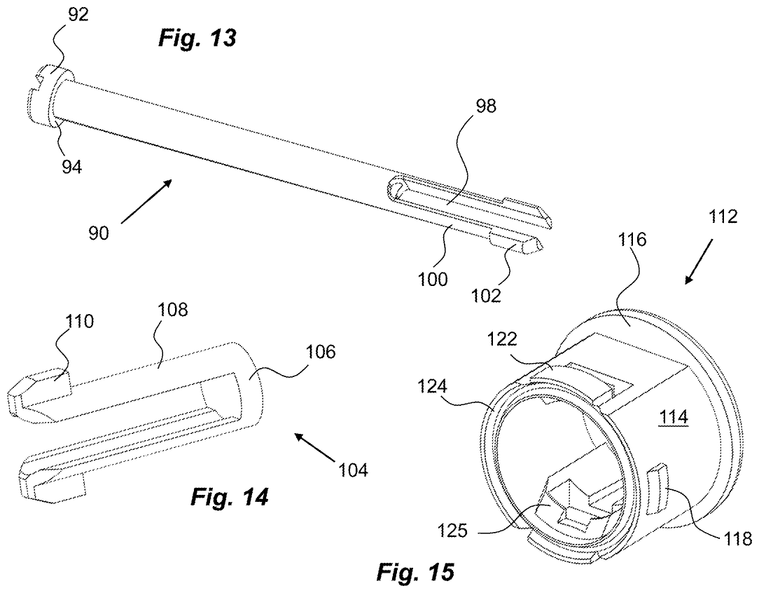

The device is further provided with a plunger rod 90, FIG. 13, having an elongated cylindrical body, that could be solid or hollow, and has an enlargement 92 at its front end. The rear surface of the enlargement is formed with a groove as a seat 94 for a drive spring 96, FIG. 1. This prevents the steel wire end of the drive spring to slip out radially and come in contact with the inside wall of the glass syringe 24. The rear section of the plunger rod 90 is arranged with an elongated slit 98, forming two flexible arms 100. The slit 98 has a width that generally corresponds to the thickness of the safety pin 88. The free ends of the arms 100 are arranged with outwardly extending protrusions or hooks 102. The forward end surfaces of the hooks 102 are formed as ledges that are to cooperate with the ledges 73 of the spring housing, as will be explained. The plunger rod 90 is surrounded by the drive spring 96, which rests between the seat 94 of the plunger rod and a forward directed surface of the transversal wall 70 of the spring housing 48.

The device is also arranged with a trigger 104 that is a part of an activator of the device, FIG. 14. The trigger 104 comprises a cylindrical body 106 with forwardly directed legs 108, where the free ends of the legs are arranged with outwardly directed ledges 110. The trigger is positioned such that the ledges 110 in the initial position are in contact with the outer surfaces of the plunger rod hooks 102.

The device is further provided with an end cap 112, FIG. 15, having a generally tubular body 114 and a rear end plate 116, to be attached to and cover the rear end of the body 12. The attachment is provided by protrusions 118 on the outer surface of the body 114 engaging cut-outs 120 at the rear end of the body. A user can use the end cap 112 as a part for a good grip when using the device, depending on grip preferences. The end cap 112 has a rear external surface that clearly show that it is blocked and that this is not the needle side of the device, when compared with the needle cover side of the device. The outer surface of the body 114 of the end cap 112 is provided with outwardly directed protrusions 122 in an axial direction which will fit between the guide ledges 17 of the body 12. The protrusions 122 will prevent the end cap 112 from being rotated if a user tries to twist the rear end cap 112 on the device. The end cap 112 has a forwardly directed spring seat 124 for the needle cover spring 78. The seat 124 is deep enough to prevent the coil wire end from being pushed inside the spring diameter and jam or interfere with the release sleeve 72 during the injection stroke. Moreover, the inner surface of the end cap 112 acts as a rear stop for the trigger 104. The end cap 112 is further provided with rearwardly facing ledges 125, on which ledges the ledges 66 of the spring housing 48 are resting, in which position the rear end surfaces of the spring housing 48 are resting against the inner surface of the end cap 112, whereby the spring housing 48 is locked from movement.

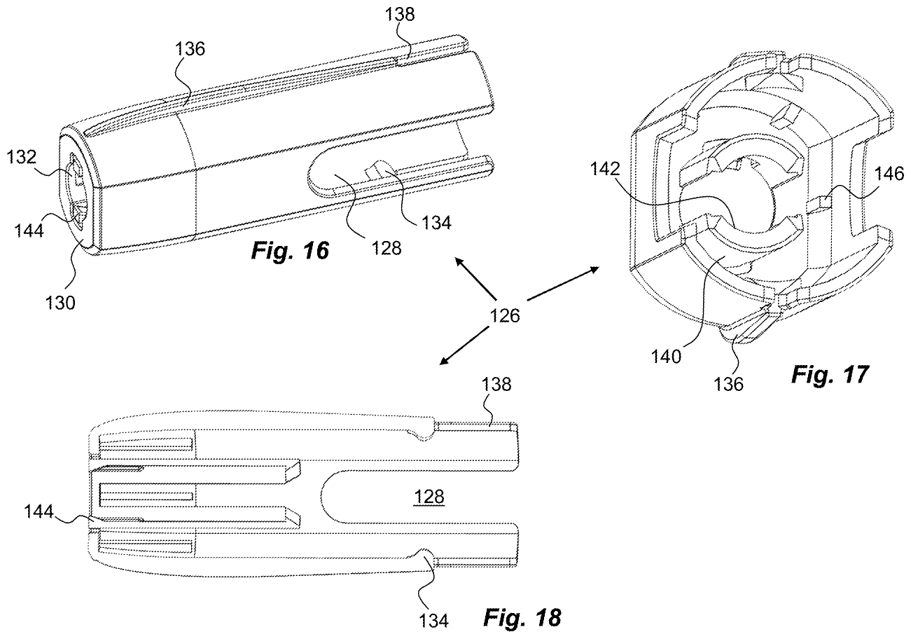

A generally tubular cap 126, FIGS. 16-18, is designed to be attached to the front end of the device as seen in FIG. 1. The cap may be provided with openings 128 on its side surface that provide access to the inspection windows in the body if the drug is not light sensitive and the user shall inspect the drug before removing the cap 126. As an alternative, the cap 126 may be designed to extend over the inspection windows 22 to block the UV-light that can degrade a drug in the syringe 24. The cap 126 has an end surface 130 with a small centre passage 132. The cap 126 is provided with protrusions 134 on its inner surface that are to interact with the recesses 15 on the outside surface of the body 12, see FIG. 1. In this regard, the cap is designed so that the cap and the protrusions can flex radially in and out of the recesses 15. The cap 126 is further provided with wings 136 on the outside which act as both roll-stop and grip feature. The cap 126 has slot-openings 138 rear of the protrusions 134, that interact with the wings 14 on the body 12. This prevents the cap 126 from being rotated during the first section of the pull-off, when the risk of rubber coring in the needle 26 of the syringe 24 is present if rubber of the RNS 29 is rotated on the needle 26. The cap 126 has inner semi-circular legs 140 with inwardly directed hooks 142 that will hook around the rear edge of the RNS 29 as seen in FIG. 1, to grip and pull off the RNS 29 from the needle 26, at cap removal. The hooks 142 will also prevent the RNS 29 from falling out of the cap 126 in the rear direction. Flexible arms 144 in the front area of the cap 126 adjacent the passage 132 will prevent the RNS 29 from falling out of the cap 126 in the front direction. Inner ribs 146 of the cap 126 will prevent the cap 126 to be pushed too long up on the outside of the body 12, abutting against the front end surface of the body 12.

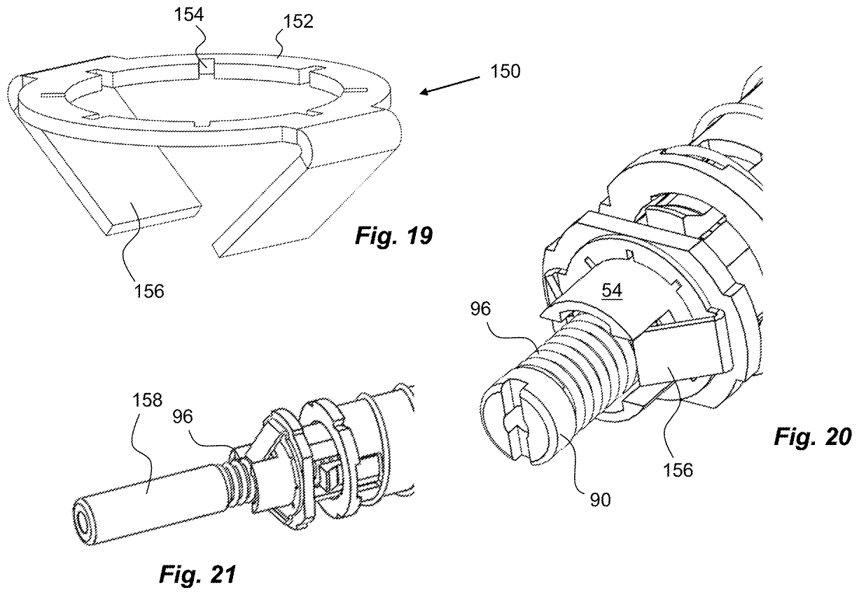

The device may further be provided with a signalling member 150 that may inform the user when the device is acting during an injection. It comprises a ring-shaped body 152, FIG. 19, that is arranged to be press-fitted onto the features 54 at the forward end of the spring housing 48 as seen in FIG. 20. The body 152 may be provided with cut-outs 154 on its inner surface, forming a lock washer function. The body is provided with tongues 156 that are formed inclined inwards the centre of the body 152. The ends of the tongues 156 are formed so as to be in contact with the coils of the drive spring 96 as seen in FIG. 20. The signalling member 150 may be made of metal or a rather rigid plastic material.

FIG. 21 shows an indication member in the form of a sheath or sleeve film 158 that may be a thin plastic tube, somewhat like a drinking straw, which is attached over the front end of the plunger rod 90 and drive spring 96. The sleeve film 158 is preferably only attached at the front end of the plunger rod 90, for example with snap fit, press fit or adhesive glue. The signal sleeve can also be placed between the signalling member 150 and the drive spring such that the rear parts of the coils are covered and the front parts are not, whereby the signalling member will be in contact with the coils only at the end of the injections stroke, creating a signal only at the end of the injection, indicating to the user that the device may be removed.

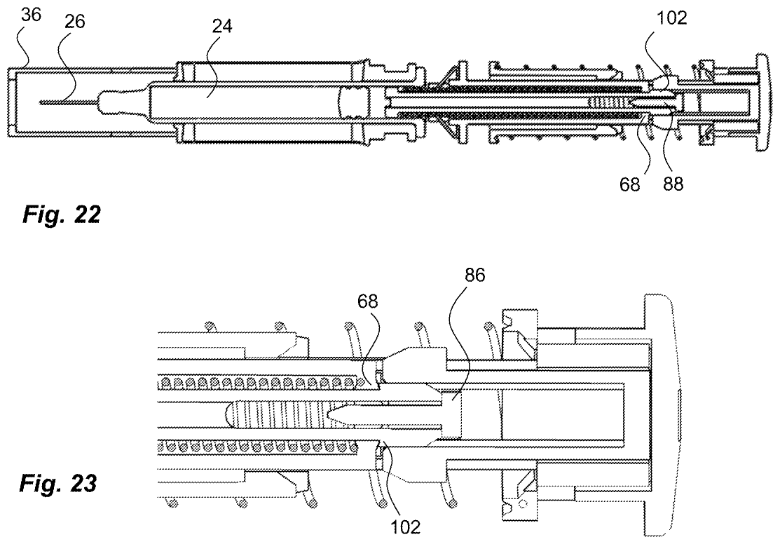

The complete device is intended to function as follows, FIGS. 22-30. The device is shown without the cap and the body, sometimes in a section view, to enhance visibility of the mechanism.

When the device is assembled, the drive spring 96 is tensioned around the plunger rod 90. The plunger rod 90 is held in the inactivated position in that its hooks 102 engage the ledges 73 of the spring housing 48. The flexible arms 100 of the plunger rod 90 are prevented from moving towards one another in a radial direction due to the safety pin 88 positioned in the slit 98, FIG. 22. The safety pin 88 ensures that the device cannot accidentally be triggered during storage, transportation, or when handled before use. Further, the beam 86 of the release sleeve 72 is positioned resting on the rear end of the plunger rod 90, FIG. 23. This ensures that the release sleeve 72 cannot travel further forward than this position at the time. The needle cover 36 is in its extended position, covering the needle 26, by the force of the needle cover spring 78.

To be used, the cap 126 is removed as described above, whereby the RNS 29 is removed from the needle 26. When the cap 126 has been removed, the needle cover 36 is free to move a short distance forward. The needle cover 36 can jangle in-between the release sleeve 72 blocking it in the rear direction and the body 12 features 21 blocking it in the front direction. An intentional feature is that the short free “jangle movement” of the needle cover 36 prior to injection, when cap 126 has been removed, is a signal that the device is active and ready to be used. When the device has been used the gap described above is reduced.

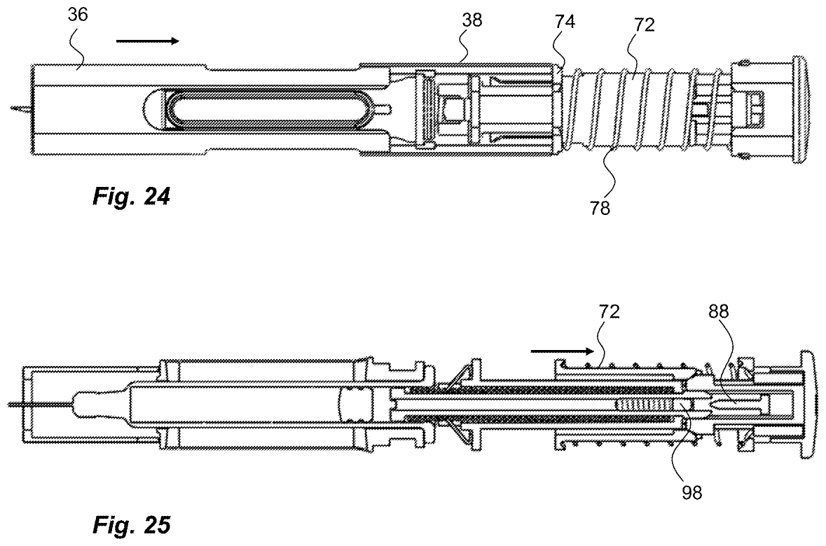

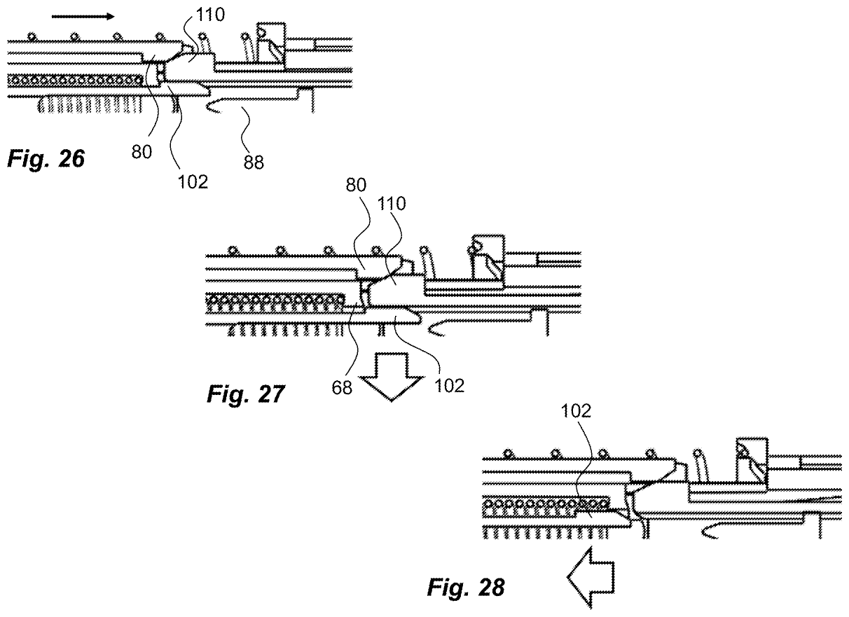

The device is placed against an injection site on the body and pressed against the injection site, causing a penetration of the needle 26. This movement causes the needle cover 36 to be pressed into the housing against the force of the needle cover spring 78, in turn pushing the release sleeve 72 in the rear direction due to the legs 38 of the needle cover 36 acting on the front plate 74, FIG. 24. The movement of the release sleeve 72 first removes the safety pin 88 from the initial position in the slit 98 between the plunger rod hooks 102, FIG. 25. The safety pin 88 is designed to leave the plunger rod 90 just before the device activates. It is to be noted that the plunger rod hooks 102 still may hang on the ledges 73 where they are installed depending on the design of contacting surfaces of the hooks 102 and the ledges 73. The ledges 82 of the release sleeve 72 will come in contact with the trigger ledges 110, FIG. 26, and at further movement of the release sleeve the ledges 82 of the release sleeve 72 will push the legs 108 of the trigger 104 in a radial direction towards the centre, FIG. 27.

As mentioned above, the ledges 82 may be provided with wedge-like surfaces 83 to achieve a smooth user feeling when reaching the last section of the activation stroke. However, this gives a “point of activation” depending on angles in the geometry. The ledges 82 guide the release sleeve 72 in a radial direction so that the tolerances and misalignment of the activation mechanism is kept to a minimum.

The radial movement of the trigger legs 108 will push the hooks 102 of the plunger rod 90 off the edges where they are installed, FIG. 28. This will activate the injection in that the drive spring 96 will move the plunger rod 90.

The contacting surfaces of the plunger rod hooks 102 and the ledges 73 of the spring housing 48 are preferably designed with a chamfer to make the hooks 102 hang on the ledges 73 by themselves, even when the safety pin 88 has been removed. This generates a positive point of activation when the plunger rod hooks 102 are mechanically pushed off the ledges 73 by the trigger 104 and the release sleeve 72, whereby successful activation does not depend on components sliding under the force of the drive spring on surfaces that offer frictional resistance. However, if one wants to minimize the force at the end of the stroke of the needle cover 36, the contacting surfaces of the plunger rod hooks 102 and the ledges 73 of the spring housing 48 can be designed with a chamfer in the opposite direction that will allow the hooks 102 to slip off the ledges 73 as soon as the safety pin 88 has been removed. On the other hand, this arrangement increases the force required earlier in the activation because the plunger rod hooks 102 squeeze the safety pin 88 and offer frictional resistance to its withdrawal. The release sleeve 72 and the trigger 104 will still push the hooks 102 together in the event that they fail to slip all the way by themselves, to achieve a fool-proof activation system.

The spring housing 48 is guiding the outside diameter of the drive spring 96 once the device is activated and the drive spring 96 expands. The rear end of the drive spring 96 is then no longer supported in the radial direction by the plunger rod 90. This support from the spring housing 48 will prevent the long drive spring 96 from buckling. The movement of the plunger rod 90 will in turn cause the stopper 28 inside the syringe 24 to move and a dose of medicament to be expelled through the needle 26. During the injection, if a signalling member 150 is used, its tongues 156 will interact with the spring coils of the drive spring 96 to generate a distinct click sound when it passes over the spring wire. The continuous clicking frequency will change during the injection stoke depending on two things: that the moving speed of the plunger rod 90 will decrease as the force from the drive spring 96 is decreasing the more it expands, and that the spring coil pitch will expand the further the plunger rod 90 moves.

During the injection, and also after an injection has been completed, the plunger rod 90 can be seen through the inspection window openings 22. The plunger rod 90 is partly covered by the coils of the drive spring 96. However, the colour of the plunger rod 90 may be used as a signal to the user, together with the visible drive spring 96, to indicate completed injection. If a sleeve film is used, it will be visible in the inspection window with a colour after use, partially or fully covering the window. The signal sleeve film 170 will hide the coils of the drive spring 96 partially or fully from being viewed in the inspection window after use of the device. The signal sleeve film 170 has an optional function to prevent the drive spring 96 from touching the glass inner wall of the syringe 24.

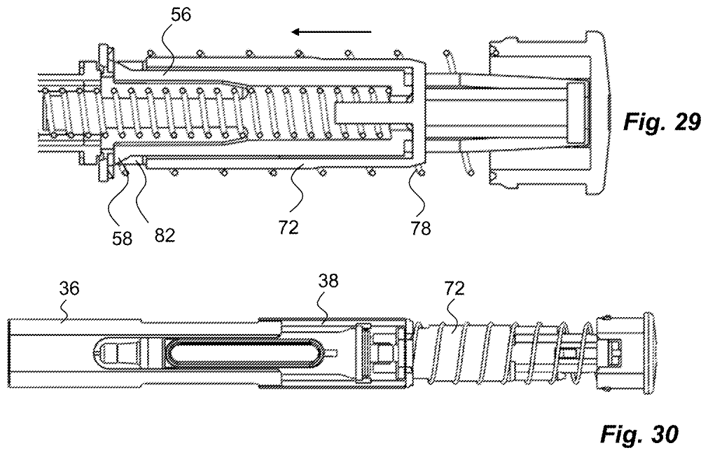

When the device is removed from the injection site, the needle cover spring 78 is pushing the release sleeve 72 forward which is pushing the needle cover 36 forward. Since the plunger rod 90 has moved from the inner wall 70 the release sleeve 72 can now travel further forward and reach a lock-out position. The needle cover spring 78 is pushing the release sleeve 72 in the forward direction wherein the arms 56 will flex inwards, and the front end of the release sleeve 72 will be moved past the lock-out hooks 58 on the spring housing 48, wherein the outwards protruding lock-out hooks 58 will enter the openings 80 of release sleeve. The needle cover 36 is thereby locked in an extended position by the release sleeve 72 via the legs 38 of the needle cover 36, FIGS. 29 and 30.

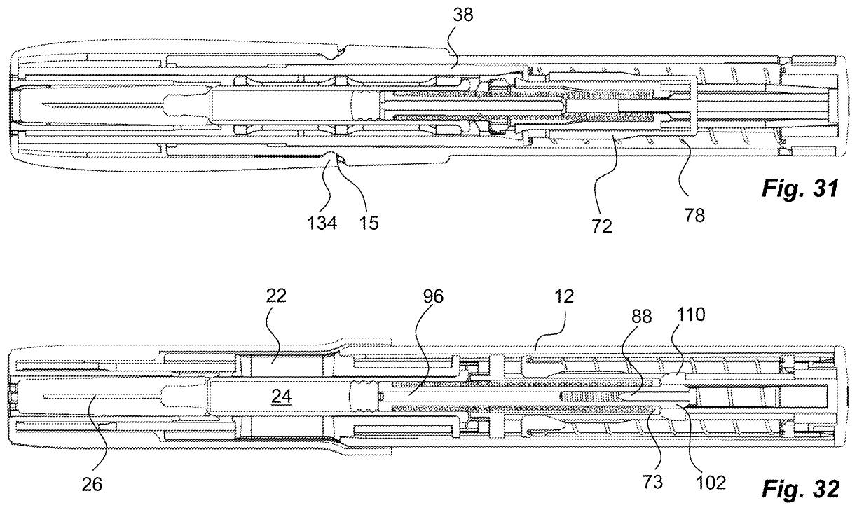

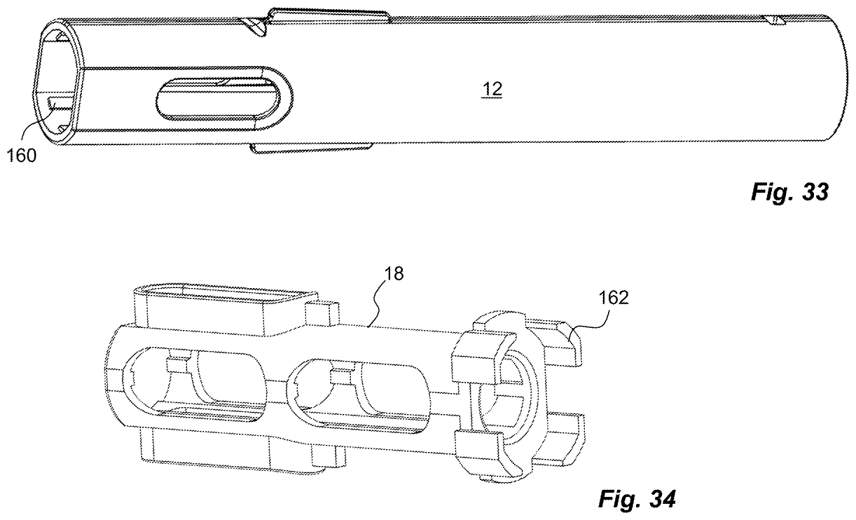

FIGS. 31 to 39 show a second embodiment of a medicament delivery device. Many of the components are the same and the functionality of the second embodiment is the same as for the first embodiment and will not be describe in detail again. However, the differences will be highlighted. The second embodiment is designed for an intramuscular injection of doses that may be larger than for the first embodiment. The body 12 of this embodiment is generally the same. However, in the front end of the body, ledges 160 are disposed on the inner surface for providing stop ledges for the needle cover 36, FIG. 33. Further, the syringe holder 18 is provided with guides 162 at its rear end for guiding a flange of a syringe, which could be a flange with cut-off portions, FIG. 34.

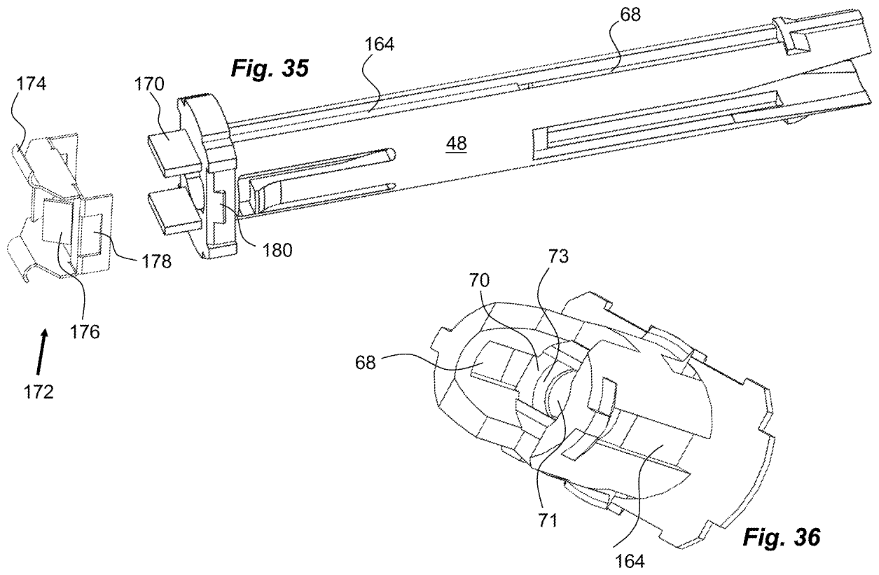

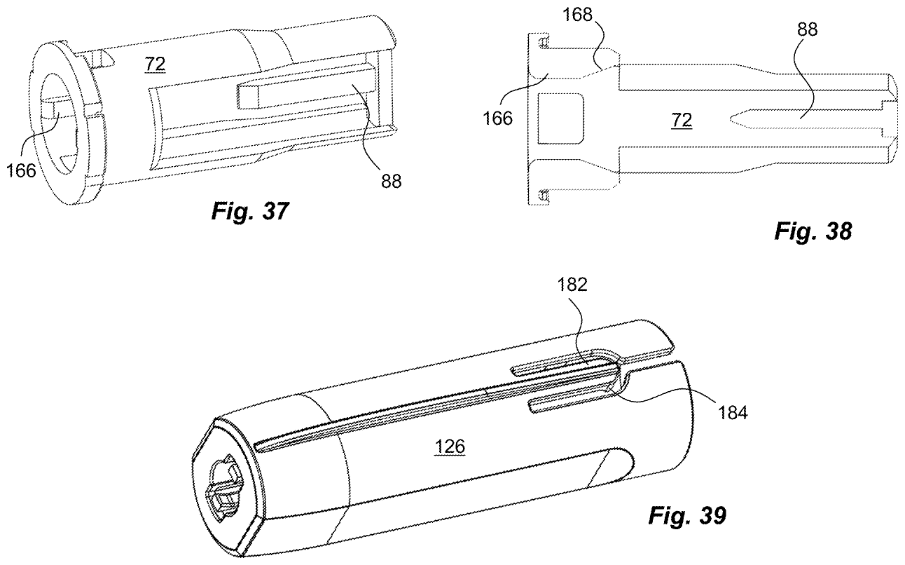

The spring housing 48 of the second embodiment has a somewhat different design. For instance, longitudinal grooves 164 are provided on its outer surface up to slits 68 in a rear section of the spring housing, FIG. 35. A transversal wall 70 is provided in the spring housing, having a central passage 71 surrounded by ledges 73 for the hooks 102 of the plunger rod 90, FIG. 36. The longitudinal grooves 164 are designed to interact with wedges 166 on an inner surface of a front section of the release sleeve 72, FIGS. 37 and 38. The wedges 166 of the release sleeve 72 are guided in the radial direction by the grooves 164 on the spring housing 48. This centres the release sleeve 72 to the plunger rod 90 and ensures that both plunger rod hooks 102 are being activated simultaneously. The wedges 166 are designed long enough so that they do not leave the grooves 164 when the trigger 104 is pressed upon, and the activation takes place. As with the previous embodiment, the wedges 166 may be provided with wedge-like surfaces 168 to achieve a smooth user feeling when reaching the last section of the activation stroke. Because the second embodiment requires a larger penetration depth, and thus a longer distance of movement of the needle cover 36 and the release sleeve 72, the release sleeve 72 is provided with a longer safety pin 88 in order to ascertain that the plunger rod 90 is released only when the full penetration depth has been reached.

The spring housing 48 is further provided with two forwardly directed plates 170, and the device may be provided with a so called click-sheet 172, FIG. 35. The click-sheet 172 comprises flexible wings 174 that are in contact with a rear surface of the flange of the syringe 24 in order to avoid a rattling syringe 24 inside the device prior to activation in that the flexible wings 174 of the click-sheet are holding the syringe 24 pushed forward. Further, the click-sheet 172 may be provided with click tongues 176 that are positioned at 90 degrees opposite the flexible wings 174. The click-tongues have the same function as the previously described tongues 156 for providing information to a user that the injection is progressing. Openings 178 in the side walls of the click-sheet 172 cooperate with protrusions 180 on the spring housing 48 for holding the component in place.

The cap may also have a somewhat different design wherein it comprises flexible arms 182, FIG. 39, in turn provided with inwardly directed protrusions 184 that fit into the recesses 15 of the body 12.

The device in its basic configuration may be able to produce a rattling sound when shaken by a user. Apart from general wide gaps in-between components to accommodate large tolerances in component manufacturing, there are two main mechanisms that could give a rattling sound.

1. The basic device, without the flex-sheet, can rattle slightly from the interface between the syringe, spring housing and syringe holder. There may be a gap of about 1 mm in-between the syringe flange and the spring housing when the syringe sits in the syringe holder seat. This gap is intentional to allow tolerance variations of the glass flange, but without the need of squeezing the syringe with a flexible member to a firm position.

2. The user can, when the cap has been pulled off, experience a rattling sound from the needle cover, body and release sleeve interface. As long as the cap is installed, the needle cover is pushed back to sit in close contact with the release sleeve and there are no extra rattling gaps. But just before use of the device, when the cap is removed and the sterile barrier of the needle is broken, the needle cover can move freely a few millimetres in-between the release sleeve and the stop surface in the body, being the extended position when the needle cover is in the locked position after use. If motivation of customer-acceptance is needed, this free movement of the needle cover just before use, a few millimetres, can be promoted as a “positive signal” to the user. It communicates that the device is “cocked” and ready to be used.

It is to be understood that the embodiment described above and shown in the drawings is to be regarded only as a non-limiting example of the invention and that it may be modified in many ways within the scope of the patent claims.

1. Medicament delivery device comprising:
a body;
a medicament container provided with a medicament delivery member disposed in the body;
a drive unit comprising a plunger rod and a drive spring (96), said drive unit operatively connected to said medicament container;
an activator operatively connected to said drive unit; and
a safety mechanism operatively connected to said plunger rod;
wherein, in an initial state of the device, said plunger rod is held with said drive spring in a tensioned state and said safety mechanism is in contact with and prevents release of said plunger rod,
wherein, when the device is pressed against an injection site, said activator acts on said safety mechanism for moving said safety mechanism out of contact with said plunger rod, and wherein said activator acts on said plunger rod for releasing said plunger rod, causing said plunger rod to act on said medicament container for delivery of medicament at said injection site.

2. Medicament delivery device according to claim 1, wherein said activator comprises a release sleeve provided with release members that are moved to act on said plunger rod for releasing said plunger rod.

3. Medicament delivery device according to claim 2, wherein said plunger rod comprises flexible arms, the flexible arms comprising protrusions that cooperate with ledges fixed in the device, and wherein said release members act on said protrusions for flexing said arms to release said plunger rod.

4. Medicament delivery device according to claim 3, wherein said safety mechanism comprises a safety pin between said flexible arms, preventing release of said plunger rod.

5. Medicament delivery device according to claim 4, wherein said safety pin is comprised in said release sleeve, the safety pin being designed to move out of contact with the arms of the plunger rod when the release members are moved to act on the protrusions of the plunger rod.

6. Medicament delivery device according to claim 3, further comprising an intermediary release member in contact with the protrusions of said plunger rod, wherein said release members of said release sleeve are pushing on push said intermediary release member for releasing said plunger rod.

7. Medicament delivery device according to claim 3, further comprising a needle cover configured to be moved into said body, thereby moving said release sleeve.

8. Medicament delivery device according to claim 7, further comprising a needle cover spring acting on said release sleeve, whereby said release sleeve and said needle cover are moved to a forward position with the needle cover covering said medicament delivery member after removal of the device from an injection site.

9. Medicament delivery device according to claim 8, wherein said release sleeve further comprises locking features configured to lock the release sleeve and the needle cover in the forward position.

10. Medicament delivery device according to claim 9, wherein said locking features comprise openings, further comprising a spring housing attached to said body, said spring housing comprising flexible locking members arranged to flex into said openings.

11. Medicament delivery device according to claim 10, wherein said spring housing is generally tubular and elongated, surrounding said drive spring for controlling buckling of said drive spring during injection.

12. Medicament delivery device according to claim 10, wherein said spring housing comprises a central passage and wherein said ledges are provided adjacent said central passage.

13. Medicament delivery device according to claim 10, wherein said spring housing further comprises support members designed to contact and support a rear surface of said medicament container.

14. Medicament delivery device according to claim 10, wherein said spring housing further comprises an audible/tactile member configured to be in contact with said drive spring and slide along said drive spring during injection, thereby creating audible/tactile information to a user.

15. Medicament delivery device according to claim 1, further comprising a syringe holder, said syringe holder comprising openings surrounded by support surfaces, wherein said body is provided with openings, wherein said support surfaces are configured to snap into said openings of said body, thereby attaching said syringe holder to said body.

16. Medicament delivery device according to claim 15, said syringe holder further comprises a seat designed for supporting a flange at a rear end of said medicament container.

17. Medicament delivery device according to claim 1, further comprising a cap attachable to a front end of the device, said cap comprising flexible hooks engaging a needle shield covering said medicament delivery member, wherein removal of said cap removes said needle shield.

18. Medicament delivery device according to claim 17, wherein said cap further comprises flexible hooks at a front area of the cap for holding said needle shield after removal.

19. Medicament delivery device according to claim 17, wherein said cap is provided with flexible members cooperating with said body for releasably holding said cap to said body.
